(19) United States
(12) Patent Application Publication
(10) Pub. No.: US 2007/0218043 A1
(43) Pub. Date: Sep. 20, 2007

(54) Use of a compound for enhancing the expression of membrane proteins on the cell surface

(75) Inventors: Michael Freissmuth, Vienna (AT); Tetyana Kirpenko, Vienna (AT); Christian Nanoff, Vienna (AT); Volodymyr M. Korkhov, Vienna (AT)

Correspondence Address: FITZPATRICK CELLA HARPER & SCINTO, 30 ROCKEFELLER PLAZA, NEW YORK, NY, 10112, US

(73) Assignee: BioDevelops Pharma Entwicklung GmBH

(21) Appl. No.: 11/650,532

(22) Filed: Jan. 5, 2007

Related U.S. Application Data
(63) Continuation of application No. 10/886,202, filed on Jul. 7, 2004, now abandoned.
(63) Continuation of application No. PCT/AT05/00251, filed on Jul. 6, 2005.

(30) Foreign Application Priority Data
Jul. 7, 2004 (AT) A 1148/2204

(51) Int. Cl. A61K 38/43; C07H 21/04; C07K 14/00; C12N 5/06

(52) U.S. Cl. 424/94.1; 435/375; 514/44; 530/350; 536/23.1

(57) ABSTRACT

The present invention is directed to the use of a compound stimulating deubiquitinating activity in a cell for the manufacture of a medicament for enhancing the expression of integral membrane proteins on the cell surface. Especially, the invention is directed to the use of such compound for the manufacture of a medicament for the treatment of a disease of condition selected from the group consisting of cystic fibrosis, diabetes insipidus, hypercholesterinaemia and long QT-syndrome-2.

### CROSS REFERENCE TO RELATED APPLICATIONS

This application is a continuation of International Patent Application No. PCT/AT2005/000251 filed on Jul. 6, 2005, which claims priority to Austrian Patent Application No. A 1148/2004 filed on Jul. 7, 2004, and U.S. patent application Ser. No. 10/886,202 filed on Jul. 7, 2004.

### BACKGROUND OF THE INVENTION

Membrane proteins, especially integral membrane proteins, have to be inserted cotranslationally into the endoplasmic reticulum. This occurs via the translocon, which is a channel formed by the Sec61-subunits. During and after synthesis of membrane proteins in the endoplasmic reticulum, they undergo a strict quality control to ensure correct folding before they are transported to their definitive site of action.

Several aspects of this quality control are incompletely understood; nevertheless it is clear that incorrectly folding of a membrane protein is sensed by the machinery of the endoplasmic reticulum (that is by chaperons, presumably). This leads to activation of ubiquitinating enzymes on the cytoplasmic side. These transfer ubiquitin to the cytoplasmic peptide chain of the incorrectly folded protein which is retrotranslocated through the Sec61 channel and degraded by the 26S proteasome (Kostova and Wolf, 2003). It has to be stressed that this scheme relies predominantly on observations that were made in Saccharomyces cervisiae. Based on several pieces of experimental evidence, it is, however, reasonable to assume that the higher eukaryotes employ a related machinery to eliminate misfolded proteins.

It has been increasingly appreciated that many human diseases can be linked to mutations, which result in the retention of the aberrant protein in the endoplasmic reticulum (ER). Cystic fibrosis is most commonly cited as the model disease: More than 1000 mutations have been identified in the gene encoding the CFTR (cystic fibrosis transmembrane conductance regulator) (Rowntree and Harris, 2003), but the majority of the patients (˜70%) have the ΔF508-mutation of the CFTR.

The resulting protein can function properly, if it reaches the plasma membrane; however, it fails to reach the plasma membrane due to an overprotective ER quality control mechanism (Pasyk and Foskett, 1995). There are many more examples that lead to defective ER-export of membrane proteins; these include mutations of the V₂-vasopressin receptor (associated with diabetes insipidus; Oksche and Rosenthal, 1998), of the LDL-receptor (resulting in hypercholesterinaemia; Hobbs et al., 1990; Jörgensen et al., 2000), or of the HERG- K⁺-channel (resulting in long QT-syndrome-2; Kupershmidt et al., 2002) etc.

It is unclear why these mutated proteins are retained and eventually degraded although they are—at least in part—functionally active (see Pasyk and Foskett, 1995). However, the available evidence suggests that the quality control machinery in the endoplasmic reticulum is overprotective.

### SUMMARY OF THE INVENTION

It is an object of the present invention to provide means for enhancing the expression of membrane proteins, especially integral membrane proteins, on the cell surface. Especially, it is an object of the present invention to provide means for enhancing the expression of a protein selected from the group consisting of CFTR (cystic fibrosis transmembrane conductance regulator), V₂-vasopressin receptor, LDL-receptor and HERG-K⁺-channel and, furthermore, to provide a medicament for the treatment of a disease or condition selected from the group consisting of cystic fibrosis, diabetes insipidus, hypercholesterinaemia and long QT-syndrome-2.

This object is achieved by the subject matter of the independent claims. Preferred embodiments are disclosed in the dependent claims.

It has been found that stimulating the deubiquitinating activity in a cell, especially by increasing the amount of deubiquitinating enzymes in the cell or stimulating them, enhances the expression of integral membrane proteins on the cell surface. Apparently, deubiquitinating enzymes are capable of decreasing the level of overprotective quality control in the endoplasmatic reticulum.

Several therapeutic concepts have been proposed that may allow to overcome the stringent quality control (see e.g. Cohen & Kelly, 2003). However, enhancing deubiquitinating activity has not yet been proposed as a strategy that would allow for enhanced surface expression of membrane proteins and mutated versions thereof.

Stimulating the deubiquitinating activity in a cell may be accomplished by any means. For example, the cell may be contacted with a compound capable of stimulating the deubiquitinating activity in the cell. Such compounds include, but are not limited to, compounds that increase the expression of deubiquitinating enzymes, compounds that suppress inhibitors of deubiquitinating enzymes, and compounds that stimulate the enzymatic activity of deubiquitinating enzymes.

Increasing the amount of deubiquitinating enzymes in the cell can be achieved especially by introducing into the cell a compound selected from the group consisting of
a deubiquitinating enzyme
a nucleic acid sequence encoding a deubiquitinating enzyme.

Especially, the cell may be transfected with an appropriate plasmid containing DNA encoding the deubiquitinating enzyme, followed by expression of the enzyme in the cell.

The ways to introduce a deubiquitinating enzyme or the nucleic acid sequence encoding the enzyme, as well as identifying suitable amounts of compound to be introduced, are known to the skilled artisan or can be determined using knowledge which is well available to the skilled artisan.

Preferably the deubiquitinating enzyme is selected from the group consisting of ubiquitin carboxy-terminal hydrolases (UCH) and ubiquitin specific proteases (USP). USPs are also being referred to as ubiquitin processing proteases (UBPs; Wing, 2003). Deubiquitinating enzymes are thiol proteases which hydrolyse the amide bond between Gly76 of ubiquitin and the substrate protein. There are two classes of deubiquitinating enzymes; the ubiquitin-specific processing protease or USP class is one of these two known classes of deubiquitinating enzymes (Papa and Hochstrasser, 1993). While the catalytic activity has been tested using artificial substrates, very little is known about their physiological substrates and thus their physiological functions. USPs have been shown to play a role in determination of cell fate (fat facets; Huang et al. (1995), transcriptional silencing (UBP3; Moazed and Johnson, D. (1996)), response to cytokines (DUBI and 2; Zhu et al., 1996) and oncogenic transformation (tre-2, USP4; Gilchrist and Baker, 2000), but the mechanistic details have remained enigmatic.

In an especially preferred embodiment, the deubiquitinating enzyme is USP-4. The sequence of murine USP-4 enzyme is, for example, disclosed in Strausberg, R. L., et al.; Proc. Natl. Acad. Sci. U.S.A. 99 (26), 16899-16903 (2002). Human USP-4 exists in two variants, cf. Puente, X. S. et al., Nat. Rev. Genet. 4 (7), 544-558 (2003).

Preferably, the medicament for enhancing expression of integral membrane proteins on the cell surface additionally comprises a compound selected from the group consisting of
a proteasome inhibitor and
a nucleic acid sequence encoding a proteasome inhibitor.

It has been found that the additional influence of a proteasome inhibitor in combination with deubiquitinating enzymes amounts to an even more significant expression of the membrane proteins on the cell surface. The fact that proteasome inhibitors may enhance the expression of membrane proteins on the cell surface, is known as such, cf. e.g. Jensen T J et al.; Cell. 1995 Oct. 6; 83(1):129-35.

Preferably, the proteasome inhibitor is MG132. MG132 is a tripeptidaldehyde having the structure leucyl-leucyl-norleucinal (LLnL).

Even more preferably, the proteasome inhibitor is Bortezomib and/or a pharmaceutically acceptable salt or ester thereof. Bortezomib (N-(2-pyrazine)carbonyl-L-phenylalanine-L-leucine-boronic acid) is a known anti-cancer agent with proteasome-inhibiting activity (EP 0 788 360 A, EP 1 123 412 A, WO 04/156854).

While proteasome inhibitors such as MG132 have been found to cause cell apoptosis even at very small administration dosage, it has surprisingly been found that there is a therapeutic window for administering Bortezomib, whereby expression of membrane proteins such as CFTR or its most common ΔF508-mutation is enhanced whilst no increased cell mortality is observed. In the case of HEK293 cells, this therapeutical window is between 1 nM and 100 nM Bortezomib, preferably from 3 nM to 10 nM. The skilled artisan can easily adapt the pharmaceutically acceptable dosis of Bortezomib depending on the disease to be treated.

The method of the present invention enables especially expression of a protein selected from the group consisting of CFTR (cystic fibrosis transmembrane conductance regulator), V₂-vasopressin receptor, LDL-receptor and HERG-K⁺-channel.

Furthermore the method of the present invention can be used for the treatment of conditions or diseases related to or associated with the lack of expression of membrane proteins on the cell surface.

Especially, the method of the present invention enables treatment of a disease or condition selected from the group consisting of cystic fibrosis, diabetes insipidus, hypercholesterinaemia and long QT-syndrome-2.

The present invention is also directed to a pharmaceutical composition, comprising a therapeutically effective amount of a compound stimulating deubiquitinating activity in a cell.

Preferably, said compound is selected from the group consisting of
a deubiquitinating enzyme
a nucleic acid sequence encoding a deubiquitinating enzyme.

Furthermore, preferably the pharmaceutical composition according to the present invention additionally comprises a therapeutically effective amount of a compound selected from the group consisting of
a proteasome inhibitor and
a nucleic acid sequence encoding a proteasome inhibitor.

### BRIEF DESCRIPTION OF THE FIGURES

FIG. 1 shows coexpression of A_{2A}-receptor and USP4 in HEK293 cells:

HEK293 cells were transiently transfected with the following sets of plasmids:

CFP-tagged A_{2A}-receptor (=A_{2A}R) (FIG. A, E); CFP-tagged A_{2A}R and GFP-tagged USP4 (FIG. B, F); CFP-tagged A_{2A}R(1-311) (FIG. C); CFP-tagged A_{2A}R(1-311) GFP-tagged USP4 (FIG. D).

Cells were incubated in the presence of the proteasome inhibitor MG132 (50 μM) for 3 h (FIG. E,F). Images were captured 24 h later with the appropriate filter settings. The experiments were carried out three times with comparable results.

FIG. 2 shows deubiquitination of the A_{2A}-receptor by USP4:

Immunoprecipitation of the A_{2A}-receptor (A_{2A}R) was carried out from HEK293 cells, transiently transfected with the following sets of plasmids:

Flag-tagged A_{2A}R, HA-tagged ubiquitin (lanes 1, 2); Flag-tagged A_{2A}R, HA-tagged ubiquitin and GFP-tagged USP4 (lanes 4,5); GFP-tagged USP4 and/or HA-tagged ubiquitin (lanes 6, 3=control lanes).

Cells were collected 48 h after transfection and membrane preparation, immunoprecipitation were done as described below. After the electrophoretic transfer, membranes with proteins were stained with anti-Flag antibody (1:500 dilution) to reveal A_{2A}-receptor immunoreactivity (upper panel), than stripped for 30 min at 50 oC and incubated with anti-HA antibody to stain ubiquitin (lower panel). Data are from a representative experiment that was reproduced 3 times.

FIG. 3A shows saturation isotherms for specific binding of [³H]ZM241385 to membranes from transiently transfected HEK293 cells expressing the full-length A_{2A} receptor:

Membranes were prepared from HEK293 cells transfected with plasmids driving the expression of the full-length Flag-tagged A_{2A}-receptor and enhanced green fluorescent protein (pEGFP) or the full-length A_{2A}-receptor and GFP tagged USP4 (=UBP4=ENP-GFP); these membranes were incubated in buffer containing the indicated concentrations of [³H]ZM241385 in the presence of 100 μM GTPgS. Data A&B are means from duplicate determinations in a representative experiment which was repeated three times (the mean parameters are shown in tabulated form).

FIG. 3B shows saturation curves for specific binding of [³H]ZM241385 to membranes from transiently transfected HEK 293 cells expressing the truncated versions of the A_{2A}-receptor [A_{2A}R(1-311) and A_{2A}R(1-360)] with or without USP4. Assay conditions were as described for FIG. 3A.

FIG. 3C shows the summary of Bₘₐₓ values from Panels A & B and saturation experiments done with membranes of cells that had been incubated for 3 h in the absence and presence of the proteasome inhibitor MG132 (50 μM):

Results are means±SD from 4 independent experiments that were carried out in parallel and done with duplicate determinations. Asterisk indicates a significant difference from the full length A_{2A}R at p=0.001 (unpaired t-test):

FIG. 4 shows the stimulation of cAMP accumulation in transiently transfected HEK293 cells:

Cells expressing solely the full-length A_{2A}-receptor (circles) or the combination of A_{2A}-receptor and USP4 (triangles) were seeded in 6-well dishes, the cellular adenine nucleotide pool was metabolically prelabeled for 16 h with [³H]adenine. After a preincubation of 30 min in fresh medium containing adenosine deaminase (2 U/ml), cAMP production was stimulated by the indicated concentrations of the A_{2A}-selective agonist CGS 21680. Data are means±SD from 4 independent experiments that were done in triplicate; in each individual experiment, the receptor alone and cotransfected with USP4 were always assayed in parallel.

FIG. 5 shows saturation curves for specific binding of [³H]ZM241385 to membranes from PC12 cells (that endogenously express the A_{2A}-receptor):

Membranes were prepared from PC12 cells, which had been incubated in the presence or in the absence of 50 μM MG132 or 100 μM chloroquine for 3 h, and were incubated in buffer containing the indicated concentrations of [³H]ZM241385 in the presence of 100 μM GTPγS.

FIG. 6 shows immunoblots of membranes from cells transfected with GFP-tagged CFTR and CFTR-Δ508, respectively, and having undergone different treatments.

FIGS. 7a, 7b and 7c, respectively, show the result of fluorescence activated cell sorting (FACS)-monitoring of the expression of GFP-tagged CFTR from HEK293 cells.

FIGS. 8a, 8b and 8c, respectively, show the result of FACS-monitoring of the expression of GFP-tagged CFTR-Δ508 from HEK293 cells.

FIGS. 9 and 10 show the comparison of expression of GFP-tagged CFTR-Δ508 from HEK293 cells which have not been co-transfected with USP-4 (FIG. 9) and cells which have been co-transfected with USP-4 (FIG. 10).

FIG. 11 shows the effect of 10 nM Bortezomib on the expression of GFP-tagged CFTR-Δ508 from HEK293 cells.

FIG. 12 shows the effect of 100 nM Bortezomib on the expression of GFP-tagged CFTR-Δ508 from HEK293 cells.

FIG. 13 shows the effect of 1 μm Bortezomib on the expression of GFP-tagged CFTR-Δ508 from HEK293 cells.

FIG. 14 shows the effect of 1 μm MG 132 on the expression of GFP-tagged CFTR-Δ508 from HEK293 cells.

### DETAILED DESCRIPTION OF THE INVENTION

### Examples

In Example 1, the A_{2A}-adenosine receptor was employed as a model protein for the following reasons:

(i) The A_{2A}-adenosine receptor is a prototypical G protein-coupled receptor and thus a representative of a class of >1000 receptors (many of which are of obvious therapeutic interest because they serve as drug targets).

(ii) G protein-coupled receptors have been documented to incur a folding problem; in other words, a large portion of newly synthesized protein (≧50%) is subject to degradation in the endoplasmic reticulum and does not reach the plasma membrane (Petaja-Repo et al., 2000 & 2001; Pankevych et al., 2003). This is similar to the situation with many other membrane proteins with multiple transmembrane spans, specifically with CFTR (Jensen et al., 1995; Rowntree and Harris, 2003).

(iii) There is at least one disease where mutations cause retention of a G protein-coupled receptor in the endoplasmic reticulum: In some instances, diabetes insipidus results from point mutations of the gene encoding the V₂-vasopressin receptor that can be linked to ER-retention of the receptor (Oksche and Rosenthal, 1998).

In Example 2, the effect of USP-4, MG 132 and Bortezomib, respectively, on the expression of the ΔF508-mutation of CFTR was examined.

Materials and Methods

Radioligand Binding Assays:

Membranes (100 μg/assay) that had been prepared from PC12 cells or HEK293 cells transiently transfected with the appropriate plasmids were incubated in a final volume of 0.3 ml containing 50 mM Tris.HCl (pH 8.0), 1 mM EDTA, 5 mM MgCl2, 8 μg/ml adenosine deaminase and concentrations of [³H]ZM241385 (specific activity ˜20 Ci/mmol) covering the range of 0.2 to 20 nM in the presence of 100 μM GTPγS (Klinger et al., 2002). After 60 min at room temperature, the reaction was terminated by rapid filtration over glass fiber filters. Nonspecific binding was determined in the presence of 10 μM XAC and amounted to 40% at the highest concentration of [³H]ZM241385. The data points were fitted by non-linear regression to the equation describing a rectangular hyperbola. Assays were performed in duplicate.

Agonist Mediated Cellular Camp Accumulation:

Cells were grown in 6-well plates. The adenine nucleotide pool was metabolically labelled by incubating confluent monolayers for 16 h with [³H]adenine (1 μCi/well) as described (Kudlacek et al. 2001). After the preincubation, fresh medium was added that contained 100 μM RO201724 (a phosphodiesterase inhibitor) and adenosine deaminase (2 U/ml) to remove any endogenously produced adenosine. After 1 h, cAMP formation was stimulated by the A_{2A}-selective agonist CGS21680 (1 nM to 1 μM) for 15 min and the reaction was stopped by adding 2.5% perchloric acid with 100 μM cAMP (1 ml/dish). The supernatant (0.9 ml) was aspirated, neutralized with 100 μl of 0.4 M KOH, and diluted with 1.5 ml 50 mM Tris-HCl, pH 8.0. [³H]cAMP was isolated by sequential chromatography on Dowex AG 50W-X4 and neutral alumina columns (Salomon (1991). Assays were performed in triplicate.

Immunoprecipitation of the Epitope-Tagged A_{2A}-Adenosine Receptor:

HEK293 cells stably expressing FLAG-tagged A_{2A}-adenosine receptor were washed three times with phosphate buffered saline; subsequently, the membranes were solubilized in ice cold lysis buffer [50 mM Tris.HCl, pH 7.5, 1 mM EDTA, 150 mM NaCl containing 1% Nonidet P-40 (vol/vol), protease inhibitors (Complete, Roche Molecular Biochemicals) and, where indicated, 10 mM N-ethylmaleimide (NEM)] for 1 h on ice. The insoluble material was collected by centrifugation at 16,000×g for 10 min at 4° C. The supernatant was processed for immunoprecipitation, each step of which was conducted with constant rotation at 4° C. Then 40 μl of a 50% (vol/vol) suspension of Anti-Flag M2 Affinity Gel (Sigma Chemical) was added and the sample was incubated overnight. The beads were collected by centrifugation and washed three times in 1 mL Tris-buffered saline. Immune complexes were dissociated in SDS-polyacrylamide sample buffer containing 20 mM dithiothreitol by incubation for 1 h at 37° C. or, alternatively, for 5 min at 95° C. Proteins were transferred to nitrocellulose membranes (Immobilon-P, Millipore) by using a semidry transfer system; immunodetection was achieved by using monoclonal peroxidase-conjugated anti-FLAG and anti-HA antibodies to detect the FLAG epitope of the A_{2A}R and the HA-epitope of ubiquitin respectively. The GFP moiety in USP4 was detected with an anti-GFP antiserum (Living colors A.v.) and a horseradish peroxidase conjugated anti-rabbit IgG secondary antibody. The immunoreactive bands were developed with the enhanced chemiluminescence detection kit (Pierce SuperSignal).

Fluorescent Microscopy:

Transiently transfected HEK-293 cells were investigated 1 day after transfection on an inverted epifluorescence microscope (Zeiss Axiovert 200M) using a 63-fold oil immersion objective and filter sets, which discriminate between CFP and YFP fluorescence (Chroma Technology Corp.; Brattleboro, Vt.). Images were captured with a cooled CCD-Camera (COOlSNAP fx; Photometrics, Roper Scientific, Tucson, Ariz.) and stored in and processed with MetaSeries software (release 4.6 Metafluor and Metamorph; Universal Imaging).

Immunoblot for CFTR and CFTR-ΔF508 Expressed in HEK293 Cells

HEK293 cells (1*10⁶ cells) were transfected with plasmids encoding CFTR or CFTR-ΔF508 (GFP-tagged) and/or co-transfected with effector plasmids. After 16 h, the cells were treated with the varying concentrations of compounds. After 24 h, the cells were harvested in phosphate-buffered saline, lysed by a freeze-thaw cycle and homogenized by sonication. The homogenate was resuspended in reducing Laemmli sample buffer (50 mM Tris.HCl, pH 6.8, 20% glycerol, 0.1% bromphenol blue, 2% SDS and 20 mM dithiothreitol); aliquots (15% of the original culture) were resolved on a denaturing polyacrylamide gel (monomer concentration in the stacking gel and in the running gel 4 and 8% respectively) and electrophoretically transferred to a nitrocellulose membrane. Immunodetection was done with an antiserum directed against GFP as the primary antibody and an anti-rabbit IgG coupled to horseradish peroxidase as the secondary antibody. Immunoreactive bands were revealed by enhanced chemiluminescence (ECL kit, Super Signal Pierce).

Fluorescence Activated Cell Sorting (FACS)

Cultured HEK293 cells were transfected with plasmids encoding CFTR or CFTR-ΔF508 (GFP-tagged) and/or co-transfected with plasmids encoding USP4 (or an appropriate control plasmid) by using the CaPO₄-precipitation method. Sixteen hours after transfection the cells were treated with varying concentrations of compounds. At a specific time point (here 24 h) the cells are trypsinized, fixed in ethanol, permeabilized and stained with propidium iodide (PI). The stained cells are subjected to FACS analysis

Results

### Example 1

USP4 Enhances the Cell Surface Expression of the A_{2A}-Adenosine Receptor

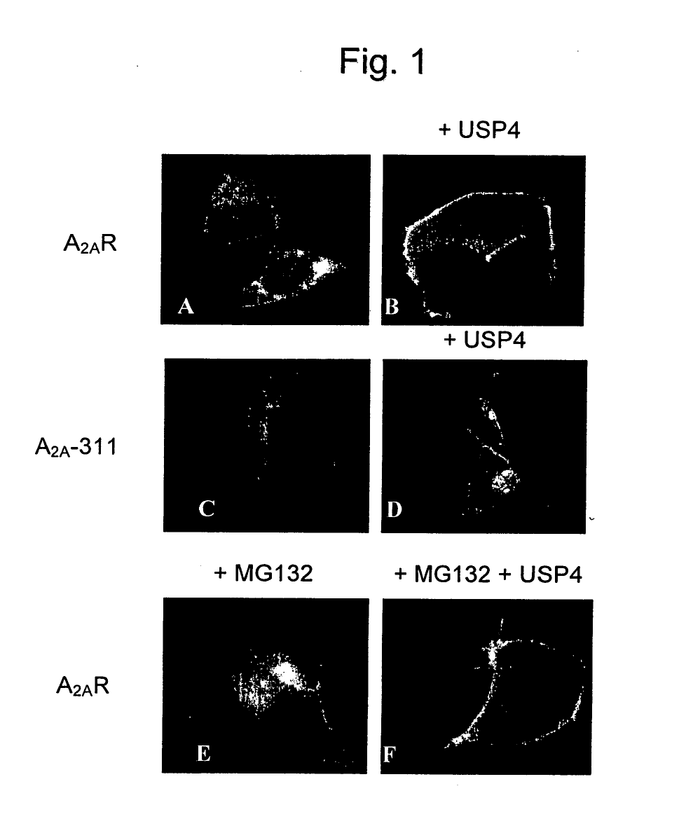

In order to visualize the A_{2A}-adenosine receptor in living cells, the receptor was tagged on its carboxyl terminus with the cyan-fluorescent protein (CFP, a spectrally shifted variant of the green fluorescent protein of Aequoria Victoria). This receptor binds ligands and activates its downstream signalling cascade in a manner indistinguishable from the untagged receptor (data not shown). Fluorescent microscopy revealed that, when expressed in HEK293 cells, a large portion of the receptor accumulates within the cell (FIG. 1A).

If the cells are cotransfected with a plasmid driving the expression of the deubiquinating enzyme USP4, the fluorescently tagged A_{2A}-adenosine receptor was found predominantly at the plasma membrane (FIG. 1B).

In the current model, quality control in the endoplasmic reticulum is thought to require ubiquitination of the carboxyl terminus (Kostova and Wolf, 2003). Therefore, it was investigated whether a truncation of the carboxyl terminus of the A_{2A}-receptor ought to render the receptor insensitive to the action of USP4. This was the case: a comparison of FIG. 1C and FIG. 1D shows that the absence and presence of USP4 does not affect the portion of fluorescent receptor that is trapped within the cell.

Finally, it was investigated whether inhibition of proteosomal degradation would, furthermore, relax quality control and thus allow the receptor to escape from the endoplasmic reticulum. The addition of the proteasome inhibitor MG132 did, in fact, augment the amount of receptor at the cell surface (cf. FIG. 1E and FIG. 1A); in the presence of both, USP4 and MG132, essentially all of the receptor was found at the cell surface (FIG. 1F).

Coexpression of USP4 Results in the Accumulation of Deubiquitinated A_{2A}-Receptor

In order to show that USP4 utilized the A_{2A}-receptor as substrate, HEK293 cells were transiently cotransfected with plasmids encoding for the Flag-tagged A_{2A}-adenosine receptor, HA-tagged ubiquitin and GFP-tagged USP4.

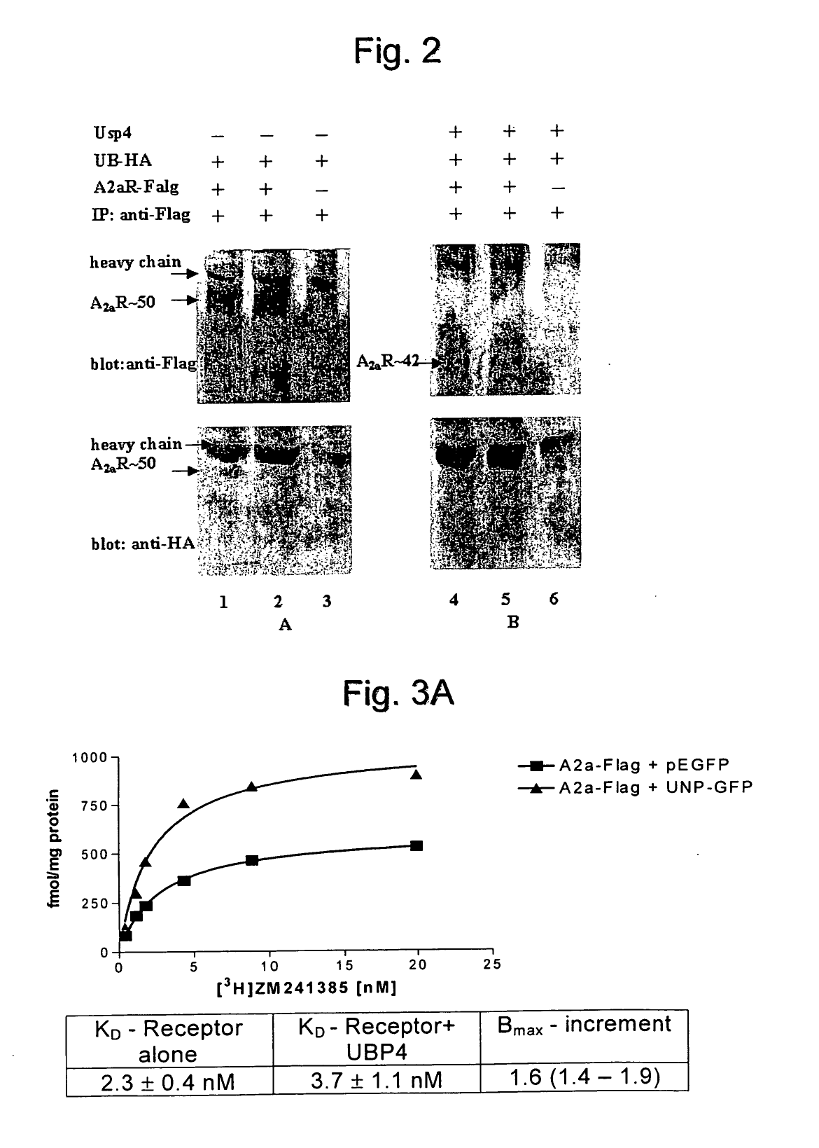

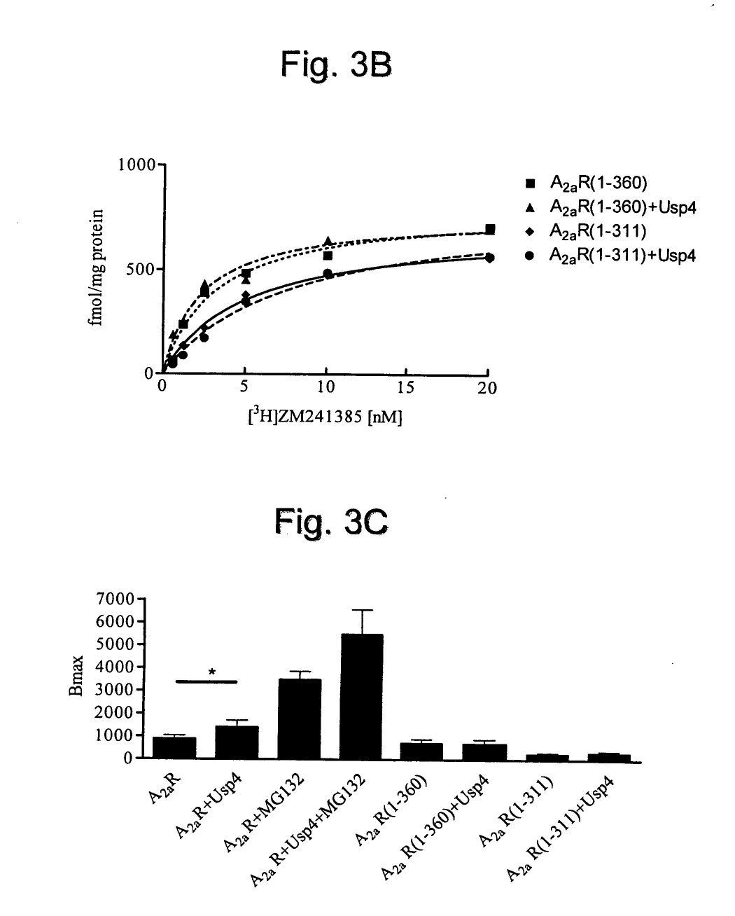

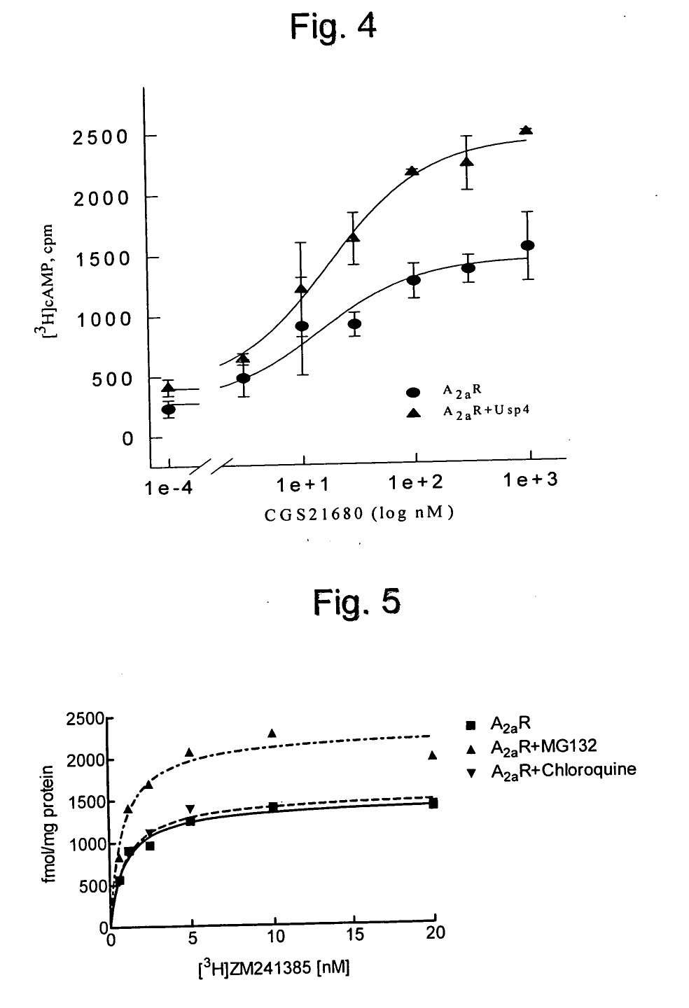

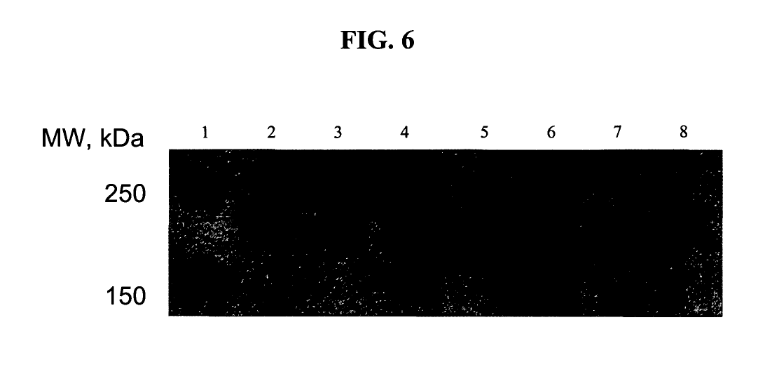

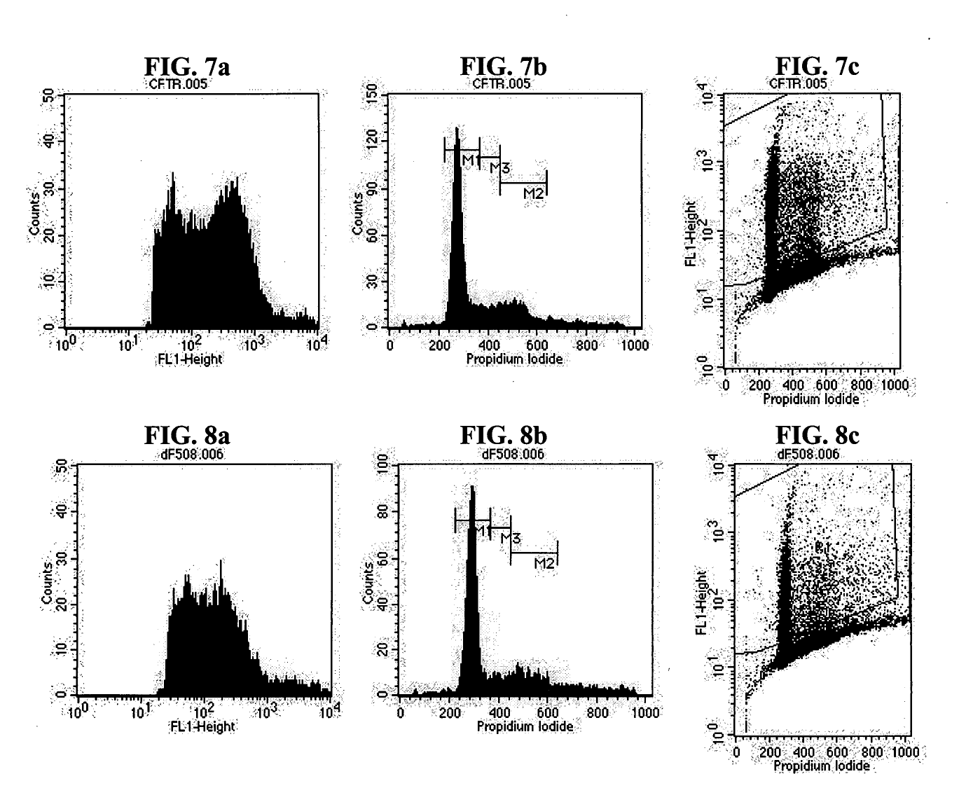

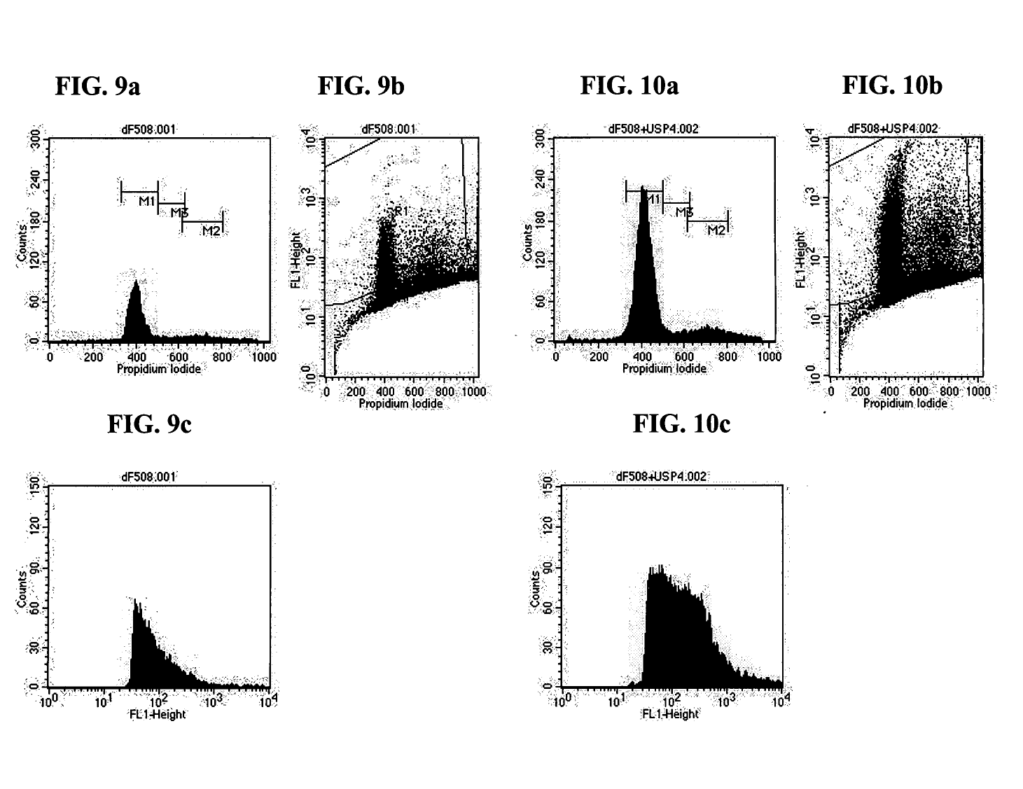

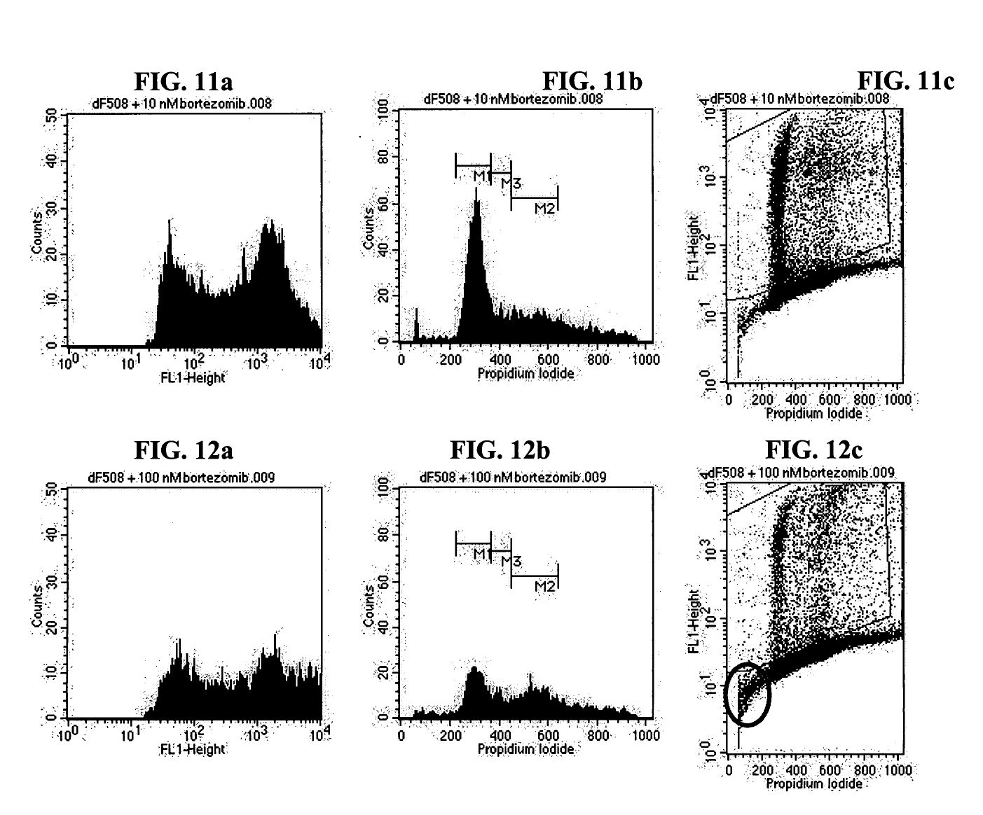

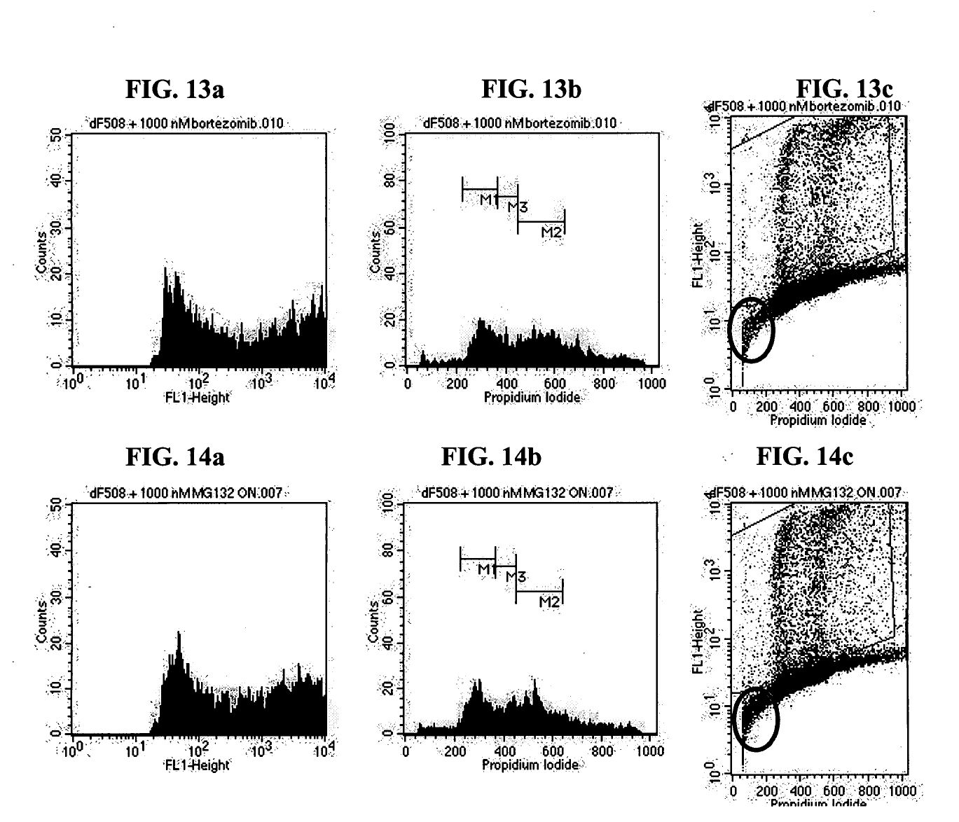

The A_{2A}-adenosine receptor was immunoprecipitated with anti-Flag antibodies from detergent lysates of cells that either coexpressed only HA-tagged ubiquitin (FIG. 2A, lanes 1,2) or the combination of HA-tagged ubiquitin and USP4 (FIG. 2B, lanes 4,5): Receptor bands were detected with anti-Flag antibody (blots shown on top); in the absence of USP4, the FLAG-reactive immunostaining was seen in the range of ˜48-50 kDa (FIG. 2A top, lanes 1,2); in the presence of USP4, the FLAG-tagged receptor migrated at ˜40-42 kDa (FIG. 2B top, lanes 1,2).

Lanes 3 and 6 represent the negative controls, that is immunoprecipitation was carried out with cellular lysates that lacked the A_{2A}-adenosine receptor but contained HA-tagged ubiquitin and—in lane 6—USP4. Regardless of the conditions, immunoreactivity was neither recovered in the ˜40-42 kDa nor in the ˜48-50 kDa range. Thus, the immunostaining was specific.

The nitrocellulose membranes were stripped and stained with anti-HA antibodies (FIGS. 2A&B, bottom blots). In cells cotransfected with the plasmids encoding the Flag tagged A_{2A}-adenosine receptor and HA-tagged ubiquitin, the HA-antibody stained a ˜48-50 kDa band. This corresponded to the ubiquitinated form of A_{2A}-receptor, because this band was also stained with the anti-HA antibody (cf. FIG. 2A top and bottom blots). In contrast, when coexpressed with USP4, the A_{2A}-receptor, which migrated as a band of 40-42 kDa (FIG. 2B, top, lanes 4&5), was not detected with the anti-HA antibody. This band, therefore represents the deubiquitinated species of the receptor.

Coexpression of USP4 Enhances the Expression of Functional A_{2A}-Receptors

As documented in FIG. 1, USP4 caused a redistribution of the CFP-tagged A_{2A}-receptor to the cell surface. It is conceivable that relaxing quality control by coexpressing USP4 allowed unfolded receptors to escape from the endoplasmatic reticulum.

In order to rule out this possibility, binding assays were performed with [³H]ZM241385, a specific and selective A_{2A}-receptor antagonist (Palmer et al., 1995). FIG. 3A shows a set of representative saturation curves for specific binding of [³H]ZM241385 to membranes from HEK293 cells that were either solely transfected with a plasmid driving the expression of (either the CFP or the FLAG-tagged) A_{2A}-receptor or of the receptor and USP4. The coexpression of USP4 (FIG. 3, red symbols) increased Bₘₐₓ but did not affect the affinity of the radioligand. This effect of USP4 depended on the carboxyl terminus of the A_{2A}-receptor, for it was not seen with the truncated forms A_{2A}-receptor-(1-311) or A_{2A}-receptor(1-360), which lack the last 100 and the last 50 amino acids respectively; representative saturation curves are shown in FIG. 3B; Bₘₐₓ averaged from several saturation experiments are shown in the bar diagram in FIG. 3C.

The model of quality control in the endoplasmatic reticulum leads to the assumption that all steps are reversible provided that the carboxyl terminus of the membrane protein has not yet been engulfed by the proteasome (Kostova and Wolf, 2003). Accordingly, it was investigated whether the action of USP4 and of proteasome inhibition is additive. This was the case. As can be seen from the average Bₘₐₓ-values summarized in FIG. 3C, sole addition of MG132 caused a pronounced increase in the amount of functional receptors, but the combined presence of both, USP4 and MG132 resulted in a dramatic increase in the number of receptors.

The A_{2A}-adenosine receptor is a prototypical Gₛ-coupled receptor, thus activation of the receptor leads to stimulation of adenylyl cyclase. The binding data showed that coexpression of USP4 increased the number of functional receptors. This conclusion was verified independently by measuring agonist-induced cellular cAMP accumulation. In cells that expressed USP4, the agonist CGS21680 elicited a larger maximum effect than in cells that only expressed the A_{2A}-adenosine receptor (FIG. 4). It should be noted that this is not a non-specific effect that can, for instance, be accounted for by an increased responsiveness of the catalytic moiety of adenylyl cyclase in the presence of USP4. Control experiments revealed that cells expressing solely the A_{2A}-receptor or the A_{2A}-receptor and USP4 did not differ in their responsiveness to forskolin.

All experiments shown so far relied on transient transfection to demonstrate the ability of USP4 to enhance the expression of the A_{2A}-receptor. Therefore, also PC12 cells, a rat pheochromocytoma cell line, in which the A_{2A}-receptor is physiologically expressed at high levels, were employed. Addition of the proteasome inhibitor MG132 also resulted in an increase in the membrane concentration of the A_{2A}-receptor (▴ in FIG. 5). In contrast, the lysosomal inhibitor chloroquine did not affect the A_{2A}-receptor levels (▾ in FIG. 5).

### Example 2

USP-4, MG 132 and Bortezomib Enhance Expression of the CFTR-ΔF508 Mutation:

In a first example, Membranes from transfected cells were prepared and immunoblotted for GFP-tagged CFTR or CFTR-ΔF508, respectively (by using an antibody directed against the fluorescent protein).

FIG. 6 shows that CFTR accumulates as a protein of ˜170 kDa, i.e. the size expected for the sum of the mass CFTR and GFP (FIG. 6, 2nd lane).

The membrane extract was also treated endoglycosidase H. The rationale for this experiment is as follows: membrane proteins are core glycosylated in the endoplasmatic reticulum. Core gylcosylation is sensitive to endoglycosidase H. If the protein has reached the Golgi (and then trafficked to the plasma membrane), it acquires additional sugar moieties and becomes resistant to endoglycosidase H. It is evident from lane 3 in FIG. 6 that endoglycosidase H treatment reduces the apparent size of CFTR; thus, the bulk of the protein is still in the ER. The following lanes examine the expression of CFTR-ΔF508 (all extracts were treated with endoglycosidase H): lane 4 is the control, that is cells expressing CFTR-ΔF508; in lanes 5, 6, 7 and 8 cells expressing CFTR-ΔF508 were treated overnight (i.e. for 16 h) with 100 nM MG132, 20 μM kifunensine, 1 μM and 100 nM bortezomib, respectively. If one compares the intensity of staining of these lanes to lane 4, it is evident that all treatments—with the exception of MG132—led to the accumulation of CFTR-ΔF508. It is also evident that 100 nM bortezomib (last lane on the right hand side) was more effective than 1 μM bortezomib (adjacent lane).

Monitoring of Expression of CFTR and CFTR-ΔF508 via FACS

Because CFTR is tagged with a fluorescent protein, expression in individual cells can be monitored by fluorescence activated cell sorting (FACS). By contrast with fluorescence microscopy (where individual cells are picked), FACS allows to survey the entire cell population. In addition, FACS has the advantage that it allows for reasonable sample throughput; finally, automation and scale-up is readily possible.

Transiently transfected HEK293 cells were fixed in ethanol 24 h after transfection as mentioned above and then stained with propidium iodide to label the DNA: the rationale was to examine the distribution of cells in the cell cycle (=to see if the expression of CFTR or of CFTR-ΔF508 was toxic or if the compounds employed killed the cells/drove them into apoptosis).

The original data set is shown on the right hand side of the figures, respectively (see e.g. FIG. 7c): the x-axis is the propidium iodide fluorescence (note that the scale is linear). The y-axis is the GFP-fluorescence (=fluorescence associated with CFTR; note that the scale is logarithmic) and each dot corresponds to a cell. The quadrangle delineates the cells that express CFTR.

One can plot the cell counts against the propidium iodide fluorescence of the transfected cells (such as shown in, for example, FIG. 7b): This gives a peak of cells (denoted by M1) that have a 2n content of DNA (G1-cells), a shoulder of cells that have a DNA content of larger than 2n (denoted by M3 and representing cells that are in S-phase) and a second peak of cells that have a DNA content of 4n (denoted by M2 and representing cells in G2 and M-phase).

The distribution of cells expressing CFTR and CFTR-ΔF508 was comparable (cf. FIG. 7, showing the result of CFTR expression and FIG. 8, showing the result of CFTR-ΔF508-expression) and comparable to that seen in untransfected cells (not shown). Thus, expression of these proteins is not toxic.

FIG. 7a and FIG. 8a, respectively, show the distribution of CFTR- or CFTR-ΔF508-associated fluorescence. It is evident that CFTR accumulates on average to higher levels: the peak is seen at 3-4*10² fluorescence units, while for CFTR-Δ508 the peak is at 10² fluorescence units.

Using the FACS assay, it was tested whether enzymatic deubiquitination by USP-4 raised the accumulation of CFTR-ΔF508; this is documented in FIGS. 9 and 10, respectively: The control situation is shown in FIG. 9: i.e. the original data set with the quadrangle defining the GFP-expressing cells=CFTR-ΔF508-expressing cells (FIG. 9b), the cell cycle distribution based on the propidium iodide fluorescence (FIG. 9a) and the level of GFP-(=CFTR-ΔF508)-associated fluorescence (FIG. 9c).

FIG. 10 shows the data set for cells cotransfected with a plasmid driving the expression of USP4: A comparison of FIG. 9c and FIG. 10c readily shows that the CFTR-ΔF508-associated fluorescence increases upon co-expression of USP4 (please note again the logarithmic scale): Under control conditions (FIG. 9c), there are essentially no cells at 10³ fluorescence units; in contrast, in the presence of USP-4, there is a substantial portion of cells containing CFTR-ΔF508-associated fluorescence at this range (FIG. 10c). Finally, if one compares the distribution of propidium iodide-fluorescence (FIG. 9a and FIG. 10a, respectively), it is evident that expression of USP4 does not affect the cell cycle distribution and does not increase the fraction of cells in the sub-2n fraction. In other words: expression of USP-4 is not toxic and does not cause apoptosis.

FIGS. 11, 12, 13 and 14 document the effect of increasing concentrations of bortezomib administered to the cells (10 nM—FIG. 11; 100 nM—FIG. 12; 1 μM—FIG. 13) and of 1 μM MG132 (FIG. 14, bottom) on the expression of CFTR-ΔF508. If one compares the CFTR-ΔF508-associated fluorescence in FIGS. 11a and 12a to the control (FIG. 8a), it is evident that the expression of CFTR is increased (the fluorescence shifts to higher intensities; please note again that the axis is logarithmic).

However, if one examines the original data set (FIG. 8c and FIG. 12c and FIG. 13c, respectively), it is evident that the number of cells with low propidium iodide fluorescence increases (marked by an ellipse in FIG. 12c and FIG. 13c) with increased Bortezomib concentration: these cells are apoptotic and have shut down translation (i.e. they do not make CFTR-ΔF508 and are hence not found in the quadrangle).

Thus if one examines the cell cycle distribution of CFTR-ΔF508 expressing cells (FIGS. 12b, 13b), one can see that cells in G1 are particularly sensitive to proteasome inhibition (the peak of the G1-cells—denoted by M1—is greatly reduced).

This is however not the case with 10 nM bortezomib (FIG. 11b): the cell cycle distribution is essentially the same as the one shown in control cells expressing CFTR-ΔF508 (FIG. 8b). Nevertheless, bortezomib substantially increases the level of CFTR-ΔF508 (FIG. 8c and FIG. 11c).

FIG. 14 demonstrates the effect of 1 μg MG 132 on HEK293 cells: As with Bortezomib at higher dosages, while MG 132 enhances CFTR-ΔF508-expression, there is also a pronounced apoptotic effect to be observed.

### REFERENCES

Cohen F E, Kelly J W. (2003) Therapeutic approaches to protein-misfolding diseases. Nature 426:905-909.
Gilchrist, C. A., Baker, R. T. (2000) Characterization of the ubiquitin-specific protease activity of the mouse/human Unp/Unph oncoprotein. Biochim Biophys Acta 1481, 297-309
Hobbs H H, Russell D W, Brown M S, Goldstein J L. (1990) The LDL receptor locus in familial hypercholesterolemia: mutational analysis of a membrane protein. Annu Rev Genet. 24:133-170.
Huang Y, Baker R T, Fischer-Vize J A. (1995) Control of cell fate by a deubiquitinating enzyme encoded by the fat facets gene. Science 270:1828-1831
Jensen T J, Loo M A, Pind S, Williams D B, Goldberg A L, Riordan J R. (1995) Multiple proteolytic systems, including the proteasome, contribute to CFTR processing. Cell 83:129-135.
Jörgensen M M, Jensen O N, Holst H U, Hansen J J, Corydon T J, Bross P, Bolund L, Gregersen N. (2000) Grp78 is involved in retention of mutant low density lipoprotein receptor protein in the endoplasmic reticulum. J. Biol. Chem. 275:33861-33868.
Klinger, M., Kuhn, M., Just, H., Stefan, E., Palmer, T., Freissmuth, M., Nanoff, C. (2002) Removal of the carboxy terminus of the A_{2A}-adenosine receptor blunts constitutive activity: Differential effect on cAMP accumulation and MAP kinase stimulation. Naunyn Schmiedeberg's Arch. Pharmacol. 366: 287-298
Kostova Z, Wolf D H. (2003) For whom the bell tolls: protein quality control of the endoplasmic reticulum and the ubiquitin-proteasome connection. EMBO J. 22:2309-2317.
Kudlacek, O., Mitterauer, T., Nanoff, C., Hohenegger, M., Tang, W.-J., Freissmuth, M., and Kleuss, C. (2001) Inhibition of adenylyl and guanylyl cyclase isoforms by the antiviral drug foscarnet. J. Biol. Chem. 276:3010-3016
Kupershmidt S, Yang T, Chanthaphaychith S, Wang Z, Towbin J A, Roden D M. (2002) Defective human Ether-a-go-go-related gene trafficking linked to an endoplasmic reticulum retention signal in the C terminus. J. Biol. Chem. 277: 27442-27448.
Moazed, D., Johnson, D. (1996) A deubiquitinating enzyme interacts with SIR4 and regulates silencing in S. cerevisiae. Cell 86: 667-677
Oksche A, Rosenthal W. (1998) The molecular basis of nephrogenic diabetes insipidus. J. Mol. Med. 76:326-337.
Palmer T M, Poucher S M, Jacobson K A, Stiles G L. (1995) ¹²⁵I-4-(2-[7-amino-2-[2-furyl][1,2,4]triazolo[2,3-a][1,3,5]triazin-5-yl-amino]ethyl)phenol, a high affinity antagonist radioligand selective for the A₂ₐ-adenosine receptor. Mol. Pharmacol. 48:970-974.
Pankevych H, Korkhov V, Freissmuth M, Nanoff C. (2003) Truncation of the A₁-adenosine receptor reveals distinct roles of the membrane-proximal carboxyl terminus in receptor folding and G protein coupling. J. Biol. Chem. 278:30283-30293
Papa F. R, Hochstrasser M. (1993) The yeast DOA4 gene encodes a deubiquitinating enzyme related to a product of the human tre-2 oncogene. Nature 366:313-319.
Pasyk E A, Foskett J K. (1995) Mutant (delta F508) cystic fibrosis transmembrane conductance regulator C1-channel is functional when retained in endoplasmic reticulum of mammalian cells. J. Biol. Chem. 270:12347-12350.
Petaja-Repo U E, Hogue M, Laperriere A, Walker P, Bouvier M. (2000) Export from the endoplasmic reticulum represents the limiting step in the maturation and cell surface expression of the human delta opioid receptor. J. Biol. Chem. 275:13727-13736.
Petaja-Repo U E, Hogue M, Laperriere A, Bhalla S, Walker P, Bouvier M. (2001) Newly synthesized human delta opioid receptors retained in the endoplasmic reticulum are retrotranslocated to the cytosol, deglycosylated, ubiquitinated, and degraded by the proteasome. J. Biol. Chem. 276:4416-4423.
Puente, X. S. et al., Human and mouse proteases: a comparative genomic approach; Nat. Rev. Genet. 4 (7), 544-558 (2003)
Rowntree R K, Harris A. (2003) The phenotypic consequences of CFTR mutations. Ann Hum Genet. 67:471-485.
Strausberg, R. L., et al.; Generation and initial analysis of more than 15,000 full-length human and mouse cDNA sequences; Proc. Natl. Acad. Sci. U.S.A. 99 (26), 16899-16903 (2002)
Wing, Simon; (2003) Deubiquitinating enzymes—the importance of driving in reverse along the ubiquitin-proteaseome pathway. IJBCB 35:590-605
Zhu Y, Pless M, Inhorn R, Mathey-Prevot B, D'Andrea A D. (1996) The murine DUB-1 gene is specifically induced by the betac subunit of interleukin-3 receptor. Mol Cell Biol. 16:4808-4817.

1. The use of a compound stimulating deubiquitinating activity in a cell for the manufacture of a medicament for enhancing the expression of membrane proteins on the cell surface.

2. The use according to claim 1, characterized in that the compound is selected from the group consisting of
a deubiquitinating enzyme and
a nucleic acid sequence encoding a deubiquitinating enzyme.

3. The use according to claim 2, characterized in that the deubiquitinating enzyme is selected from the group consisting of ubiquitin carboxy-terminal hydrolases (UCH) and ubiquitin specific proteases (USP).

4. The use according to claim 3, characterized in that the deubiquitinating enzyme is USP-4.

5. The use according to any one of the foregoing claims, characterized in that the medicament additionally comprises a compound selected from the group consisting of
a proteasome inhibitor and
a nucleic acid sequence encoding a proteasome inhibitor.

6. The use according to claim 5, characterized in that the proteasome inhibitor is MG132.

7. The use according to any one of the foregoing claims for the manufacture of a medicament for enhancing the expression of a protein selected from the group consisting of CFTR (cystic fibrosis transmembrane conductance regulator), V₂-vasopressin receptor, LDL-receptor and HERG-K⁺-channel.

8. The use according to any one of the foregoing claims for the manufacture of a medicament for the treatment of a disease or condition selected from the group consisting of cystic fibrosis, diabetes insipidus, hypercholesterinaemia and long QT-syndrome-2.

9. Pharmaceutical composition, comprising a therapeutically effective amount of a compound stimulating deubiquitinating activity in a cell.

10. Pharmaceutical composition according to claim 9, characterized in that the compound is selected from the group consisting of
a deubiquitinating enzyme
a nucleic acid sequence encoding a deubiquitinating enzyme.

11. Pharmaceutical composition according to claim 9 or 10, additionally comprising a therapeutically effective amount of a compound selected from the group consisting of
a proteasome inhibitor and
a nucleic acid sequence encoding a proteasome inhibitor.

12. A method for enhancing the expression of membrane proteins on a cell surface, comprising the step of stimulating the deubiquitinating activity in the cell.

13. Method according to claim 12, comprising the step of increasing the amount of deubiquitinating enzymes in the cell.

14. Method according to claim 13, wherein a compound selected from the group consisting of
a deubiquitinating enzyme and
a nucleic acid sequence encoding a deubiquitinating enzyme is introduced into the cell.

15. Method according to claim 14, wherein said deubiquitinating enzyme is selected from the group consisting of ubiquitin carboxy-terminal hydrolases (UCH) and ubiquitin specific proteases (USP).

16. Method according to claim 15, wherein the deubiquitinating enzyme is USP-4.

17. Method according to any one of the foregoing claims, comprising the additional step of increasing the amount of proteaseome inhibitors in the cell.

18. Method according to claim 17, wherein a compound selected from the group consisting of
a proteasome inhibitor and
a nucleic acid sequence encoding a proteasome inhibitor is introduced into the cell.

19. Method according to claim 18, wherein in the proteasome inhibitor is MG132.

20. Method according to any of claims 12 to 19, for enhancing the expression of a protein selected from the group consisting of CFTR (cystic fibrosis transmembrane conductance regulator) V₂-vasopressin receptor, LDL-receptor and HERG-K⁺-channel.

21. A method for treating a disease or condition selected from the group consisting of cystic fibrosis, diabetes insipidus, hypercholesterinaemia and long QT-syndrome-2, comprising the step of administering to a patient in need thereof a pharmaceutically effective amount of a compound selected from the group consisting of
a deubiquitinating enzyme and
a nucleic acid sequence encoding a deubiquitinating enzyme.

22. Method according to claim 21, comprising the step of additionally administering to said patient a compound selected from the group consisting of
a proteasome inhibitor and
a nucleic acid sequence encoding a proteasome inhibitor.
